(19) United States
(12) Patent Application Publication
(10) Pub. No.: US 2013/0172880 A1
(43) Pub. Date: Jul. 4, 2013

(54) RENAL NERVE MODULATION DEVICES AND METHODS FOR RENAL NERVE MODULATION

(71) Applicant: Boston Scientific Scimed, Inc., Maple Grove, MN (US)

(72) Inventor: MARTIN R. WILLARD, BURNSVILLE, MN (US)

(73) Assignee: BOSTON SCIENTIFIC SCIMED, INC., MAPLE GROVE, MN (US)

(21) Appl. No.: 13/715,906

(22) Filed: Dec. 14, 2012

Related U.S. Application Data
(60) Provisional application No. 61/581,451, filed on Dec. 29, 2011.

(51) Int. Cl. A61B 18/14

(52) U.S. Cl. CPC A61B 18/1492
USPC 606/41

(57) ABSTRACT

Medical devices and methods for making and using medical devices are disclosed. An example method may include a method for renal nerve modulation. The method may include providing a steerable medical device, advancing the steerable medical device through a vein to a position adjacent to the kidney, advancing the steerable medical device through a vessel wall of the vein and into body tissue positioned adjacent to a renal artery, and steering the steerable medical device around at least a portion of the renal artery.

### CROSS-REFERENCE TO RELATED APPLICATIONS

This application claims priority under 35 U.S.C. §119 to U.S. Provisional Application Ser. No. 61/581,451, filed Dec. 29, 2011, the entirety of which is incorporated herein by reference.

### TECHNICAL FIELD

The present disclosure pertains to medical devices, and methods for manufacturing and using medical devices. More particularly, the present disclosure pertains to methods for renal nerve modulation.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example method may include a method for renal nerve modulation. The method may include providing a steerable medical device, advancing the steerable medical device through a vein to a position adjacent to the kidney, advancing the steerable medical device through a vessel wall of the vein and into body tissue positioned adjacent to a renal artery, and steering the steerable medical device around at least a portion of the renal artery.

Another example method for renal nerve modulation may include providing a steerable ablation guidewire having one or more ablation members coupled thereto, advancing the steerable ablation guidewire through a renal vein to a position adjacent to the kidney, advancing the steerable ablation guidewire through a vessel wall of the renal vein and into body tissue positioned adjacent to a renal artery, steering the steerable ablation guidewire around at least a portion of the renal artery, and ablating renal nerves disposed adjacent to the renal artery with the one or more ablation members.

Another example method for renal nerve modulation may include providing a steerable guidewire, advancing the steerable guidewire through a renal vein to a position adjacent to the kidney, advancing the steerable guidewire through a vessel wall of the renal vein and into body tissue positioned adjacent to a renal artery, steering the steerable guidewire around at least a portion of the renal artery, and advancing an ablation catheter over the steerable guidewire and around at least the portion of the renal artery. The ablation catheter may have one or more ablation members coupled thereto. The method may also include ablating renal nerves disposed adjacent to the renal artery with the one or more ablation members.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view illustrating an example renal nerve modulation system;

FIG. 2 is a schematic view illustrating the location of the renal nerves relative to the renal artery;

FIG. 3 is a side view of a portion of an example medical device;

FIG. 4 is a plan view illustrating an example medical device extending through a renal vein and around a portion of a renal artery; and

FIG. 5 is a plan view illustrating an example medical device system extending through a renal vein and around a portion of a renal artery.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless clearly stated to the contrary.

Certain treatments may require the temporary or permanent interruption or modification of select nerve function. One example treatment is renal nerve modulation and/or ablation which is sometimes used to treat conditions related to hypertension and/or congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

Because the nerves are hard to visualize, treatment methods employing such energy sources have tended to apply the energy as a generally circumferential ring to ensure that the nerves are modulated. However, such a treatment may result in thermal injury to the vessel wall near the electrode and other undesirable side effects such as, but not limited to, blood damage, clotting, weakened vessel wall, and/or protein fouling of the electrode.

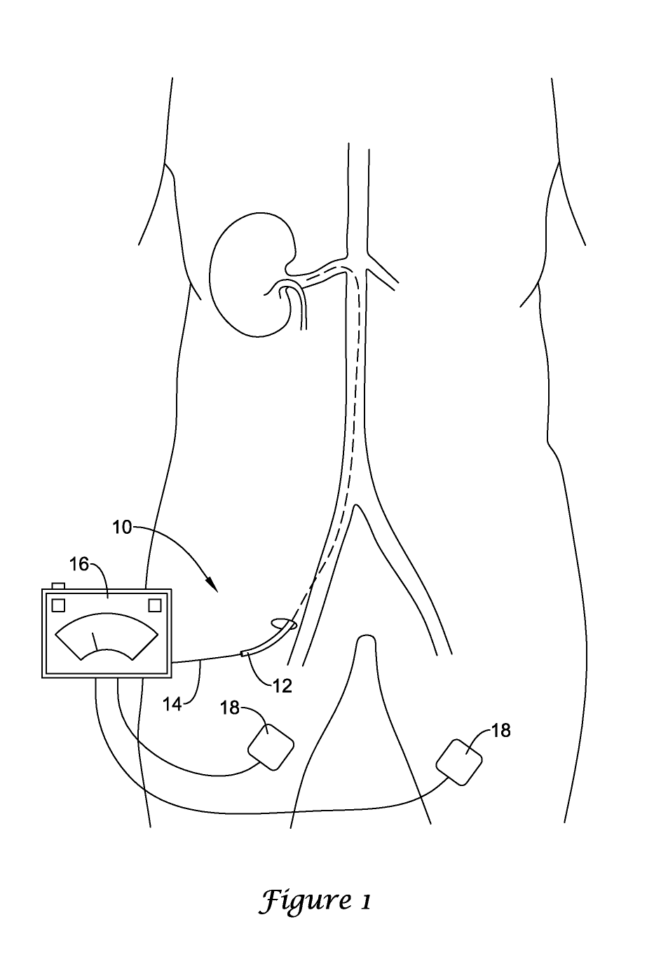

FIG. 1 is a schematic view of an example renal nerve modulation system 10 in situ. System 10 may include a renal ablation catheter 12 and one or more conductive element(s) 14 for providing power to catheter 12. A proximal end of conductive element(s) 14 may be connected to a control and power element 16, which supplies necessary electrical energy to activate one or more electrodes disposed at or near a distal end of catheter 12. When suitably activated, the electrodes are capable of ablating adjacent tissue. The terms electrode and electrodes may be considered to be equivalent to elements capable of ablating adjacent tissue in the disclosure which follows. In some instances, return electrode patches 18 may be supplied on the legs or at another conventional location on the patient's body to complete the circuit.

Control and power element 16 may include monitoring elements to monitor parameters such as power, temperature, voltage, amperage, impedance, pulse size and/or shape and other suitable parameters, with sensors mounted along catheter, as well as suitable controls for performing the desired procedure. In some embodiments, power element 16 may control a radio frequency (RF) electrode. The electrode may be configured to operate at a frequency of approximately 460 kHz. It is contemplated that any desired frequency in the RF range may be used, for example, from 450-500 kHz. It is further contemplated that additionally and/or other ablation devices may be used as desired, for example, but not limited to resistance heating, ultrasound, microwave, and laser devices and these devices may require that power be supplied by the power element 16 in a different form.

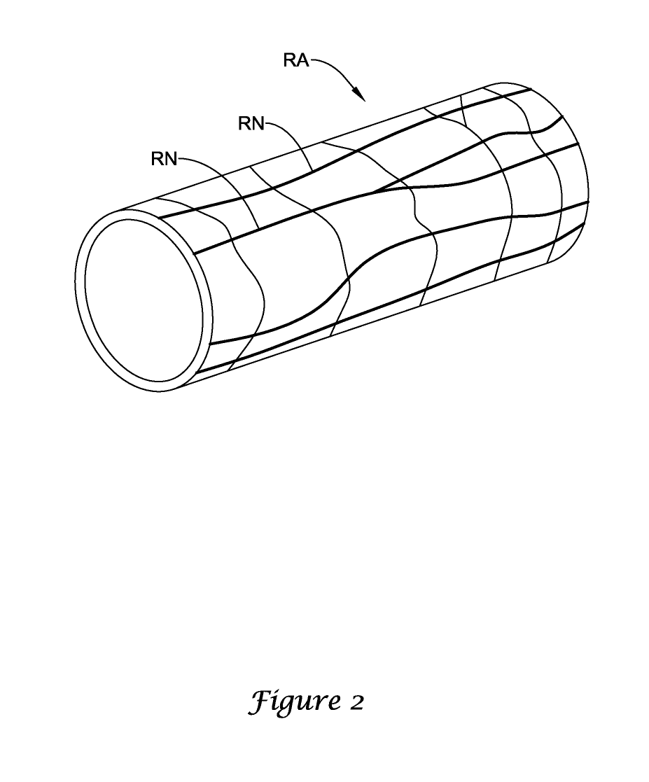

FIG. 2 illustrates a portion of the renal anatomy in greater detail. More specifically, the renal anatomy includes renal nerves RN extending longitudinally along the lengthwise dimension of renal artery RA and generally within or near the adventitia of the artery. The human renal artery wall is typically about 1 mm thick of which 0.5 mm is the adventitial layer. As will be seen in the figure, the circumferential location of the nerves at any particular axial location may not be readily predicted. Nerves RA are difficult to visualize in situ and so treatment methods may desirably rely upon ablating multiple sites to ensure nerve modulation.

While renal nerve modulation with the use of an ablation catheter or other medical device advanced into the renal artery appears to be a useful treatment strategy, such methods may have some limitations. For example, anatomical differences in the distribution of nerves about the renal artery and/or other structural differences in the nearby anatomy may lead to incomplete nerve ablation. Because of this, some treatment strategies employ multiple small ablation spots at a number of different locations. However, such an approach may require a number of catheter repositioning steps, which may complicate and/or prolong the intervention. In addition, ablation devices that ablate from within the renal artery can cause thermal injury and/or damage to the arterial wall. Furthermore, sealing the arterial access site (e.g., at the femoral artery) may pose technical challenges. Disclosed herein are methods and devices for renal nerve modulation that utilize a trans-venous approach that may alleviate some of the limitations of arterial-based interventions.

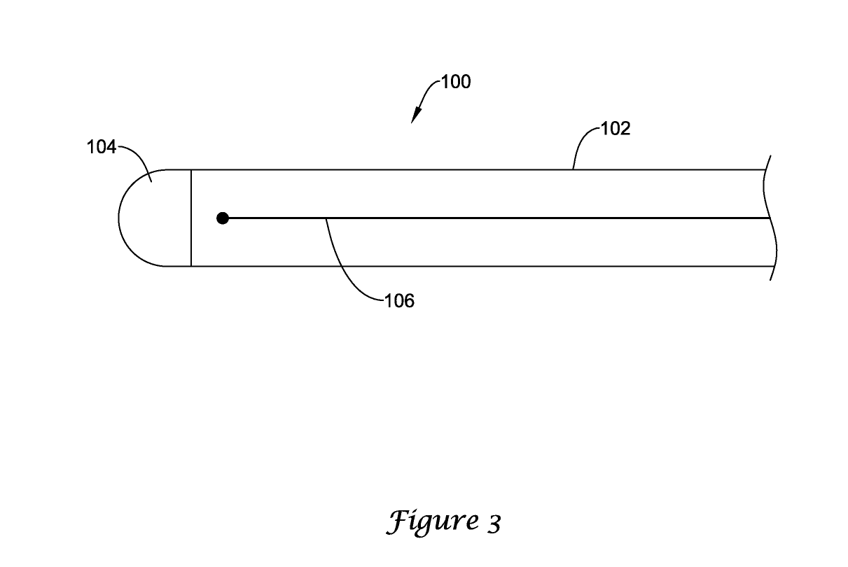

FIG. 3 illustrates a portion of an example renal nerve modulation medical device 100. It should be noted that medical device 100 is shown highly schematically. For example, medical device 100 may take the form of a steerable ablation guidewire. Other embodiments are contemplated, however, where medical device 100 may be a catheter, guidewire, or the like, or any other suitable medical device. In at least some embodiments, medical device 100 may include an elongate shaft or body 102 having one or more electrodes 104 disposed at or adjacent to the distal end of shaft 102. A pull wire 106 may be coupled to shaft 102. In general, pull wire 106 may be configured to deflect shaft 102 and/or shift shaft 102 between a generally elongated or “straight” configuration and a bent or curved configuration.

It can be appreciated that the form of medical device 100 may vary. For example, medical device 100 may include a number of different structural elements typically utilized for medical devices including, for example, tubular members, slotted tubular members, hypotubes, core wires, solder tips, spring tips, polymer tips, polymer jackets and/or sleeves, coatings, or the like. In addition, medical device 100 may include a singular electrode 104 or a plurality (e.g., two, three, four, five, six, seven, eight, or more) or array of electrodes 104 that may span a portion of the length of medical device 100. For example, the electrodes 104 may be arranged circumferentially about shaft 102 and/or may be spaced longitudinally along shaft 102. In some embodiments, electrode(s) 104 may be disposed at the distal end of shaft 102. In other embodiments, one or more of electrode(s) 104 may be disposed proximally of the distal end of shaft 102. The electrodes 104 may include RF ablation electrodes, ultrasound transducers, resistance heating electrodes, microwave electrodes, etc.

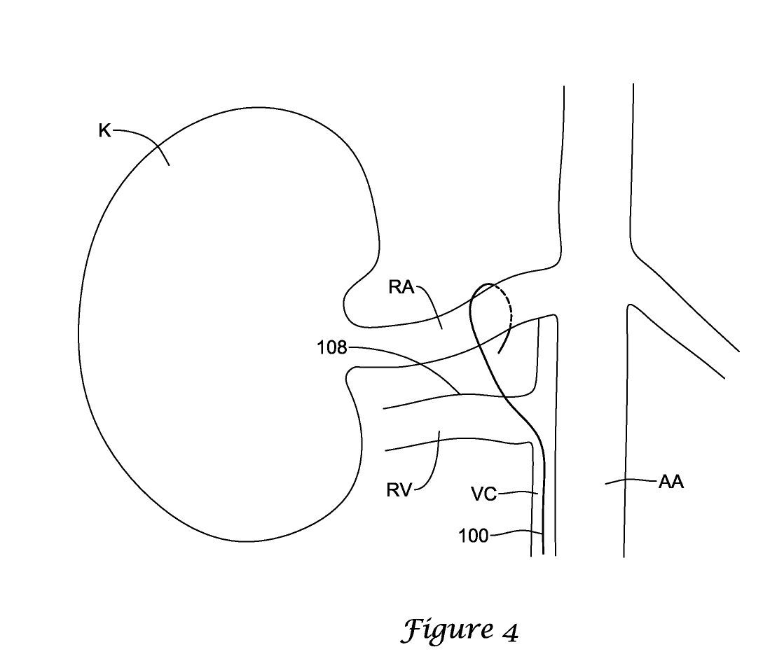

The methods disclosed herein generally include advancing medical device 100 through the venous system of the patient as illustrated in FIG. 4. For example, a clinician may advance medical device 100 through a jugular vein (not shown) and through the vena cava VC to a position adjacent to the kidney K of a patient. Other approaches may also be used. This may include advancing and/or “steering” (e.g., via pull wire 106) medical device 100 into a renal vein RV. When suitably positioned, medical device 100 can be advanced through the vessel wall 108 of the renal vein RV (or the vessel wall of the vena cava VC, as desired) and enter tissue adjacent to the renal artery RA. Medical device 100 may be further advanced and steered around the periphery of the renal artery RA. This may include disposing medical device 100 around essentially the entire circumference of the renal artery RA (e.g., complete circumferential coverage) or around a portion of the renal artery RA (e.g., partial circumferential coverage). Steering may be aided by fluoroscopic visualization and may include, for example, pull on pull wire 106 to guide medical device 100 in the desired direction.

Electrode 104 may be used to ablate tissue including renal nerves. For example, ablation can take place while medical device 100 is advanced through the vessel wall 108 of the renal vein RV, while positioned within tissue generally located between the renal vein RV and the renal artery RA, while being steered and/or advanced around the renal artery RA, while being retracted from patient, or in combinations of these times and/or processes. This may also include ablation with any one or more of electrodes 104 simultaneously or in sequence at any of these portions of the intervention.

When the desired ablation is completed, medical device 100 may be removed from the vasculature. The entry site (e.g., an opening formed in the jugular vein) can then be sealed. Because the pressure within the venous vasculature is generally lower than within arterial vasculature, sealing the entry site may be accomplished easier and may be done so without the use of typical vascular sealing devices or aids commonly used for arterial sealing.

In general, the methods disclosed herein can be performed without passing medical device 100 through the abdominal aorta AA and/or the renal artery RA. In addition to what is disclosed and/or suggested above, this trans-venous approach to renal nerve modulation may be desirable for a number of reasons. For example, the trans-venous approach may provide access to nerves that may be located around the renal artery, between the renal artery and the renal vein, around the renal vein, and adjacent to the renal vein. In addition, the total area that targeted for renal nerve modulation and/or the number of nerves that may be targeted for modulation may be increased. The trans-venous approach may reduce plaque disruption (e.g., plaque disruption that may occur when using an arterial approach), reduce the likelihood of vessel dissection (e.g., arterial dissection), reduce arterial damage, etc. Furthermore, the trans-venous approach may utilize the venous system as a heat sink for dissipating heat that may be generated during an ablation procedure.

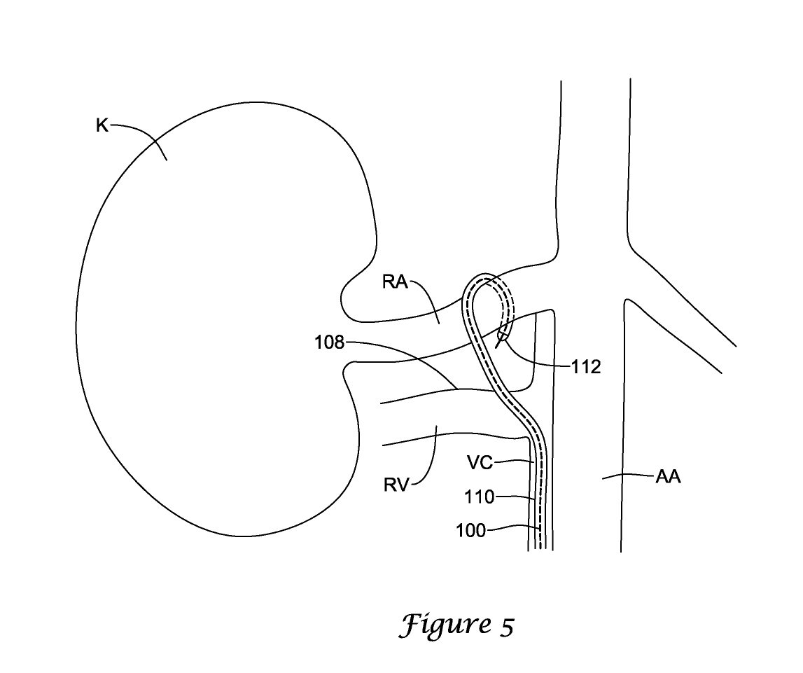

While the process described above generally includes the use of an ablation wire or guidewire (e.g., medical device 100), other methods are contemplated that may include the use of additional and/or different devices. For example, FIG. 5 schematically depicts a catheter 110 that is advanced over medical device 100. According to this embodiment, catheter 110 may be ablation catheter (e.g., including one or more ablation electrodes and/or transducers 112) that can be tracked over medical device 100 and be used to ablate target tissue (e.g., renal nerves). This may include ablation while catheter 110 is advanced through the vessel wall 108 of the renal vein RV, while positioned within tissue generally located between the renal vein RV and the renal artery RA, while being steered and/or advanced around the renal artery RA, while being refracted from patient, or in combinations of these times and/or processes. It can be appreciated that the use of ablation catheter 110 may allow the clinician to use a steerable guidewire (rather than medical device 100, which may take the form of an ablation wire). However, ablation catheter 110 can still be used in combination with medical device 100.

In at least some embodiments, catheter 110 may be steerable. For example, catheter 110 may include one or more pull wires (not shown) or other features that allow catheter 110 to be deflected during an intervention. In these embodiments, catheter 110 may be advanced through the renal vein, through the vena cava, and, if desired, into the renal vein. Thereafter, catheter 110 can be passed through the vessel wall 108 and be “steered” around at least a portion of the renal artery RA. Such procedures may be performed while advancing catheter 110 over medical device 100 or independently of medical device 100.

The materials that can be used for the various components of medical device 100 (and/or other medical devices disclosed herein) and the various shaft and/or members disclosed herein may include those commonly associated with medical devices. For simplicity purposes, the following discussion makes reference to shaft 102 and other components of medical device 100. However, this is not intended to limit the devices and methods described herein, as the discussion may be applied to other similar tubular members and/or components of tubular members or devices disclosed herein.

Shaft 102 and/or other components of medical device 100 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of shaft 102 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of medical device 100 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of medical device 100 to achieve the same result.

In some embodiments, a degree of Magnetic Resonance Imaging (MRI) compatibility is imparted into medical device 100. For example, catheter shaft 20 and/or shaft 102, or portions thereof, may be made of a material that does not substantially distort the image and create substantial artifacts (i.e., gaps in the image). Certain ferromagnetic materials, for example, may not be suitable because they may create artifacts in an MRI image. Shaft 102, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of shaft 102 that may define a generally smooth outer surface for medical device 100. In other embodiments, however, such a sheath or covering may be absent from a portion of all of medical device 100, such that shaft 102 may form the outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the medical device 100 (including, for example, the exterior surface of shaft 102) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of shaft 102 or other portions of medical device 100. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A method for renal nerve modulation, the method comprising:
providing a steerable medical device;
advancing the steerable medical device through a vein to a position adjacent to a kidney;
advancing the steerable medical device through a vessel wall of the vein and into body tissue positioned adjacent to a renal artery;
steering the steerable medical device around at least a portion of the renal artery.

2. The method of claim 1, wherein the steerable medical device includes a guidewire.

3. The method of claim 1, wherein the steerable medical device includes a catheter.

4. The method of claim 1, wherein the steerable medical device includes one or more ablation members and wherein the method further comprises ablating tissue with the one or more ablation members.

5. The method of claim 4, wherein ablating tissue with the one or more ablation members includes ablating renal nerves disposed about the renal artery.

6. The method of claim 4, wherein ablating tissue with the one or more ablation members includes ablating renal nerves disposed between the renal artery and a renal vein.

7. The method of claim 1, further comprising advancing a catheter over the steerable medical device.

8. The method of claim 7, wherein the catheter includes one or more ablation members and wherein the method further comprises ablating tissue with the one or more ablation members.

9. The method of claim 1, wherein steering the steerable medical device around at least a portion of the renal artery includes extending the steerable medical device around only a portion of the circumference of the renal artery.

10. The method of claim 1, wherein steering the steerable medical device around at least a portion of the renal artery includes extending the steerable medical device around the entire circumference of the renal artery.

11. The method of claim 1, wherein advancing the steerable medical device through a vein to a position adjacent to the kidney includes advancing the steerable medical device through a jugular vein.

12. The method of claim 1, wherein advancing the steerable medical device through a vein to a position adjacent to the kidney includes advancing the steerable medical device through the vena cava.

13. The method of claim 1, wherein advancing the steerable medical device through a vein to a position adjacent to the kidney includes forming an opening in a jugular vein, and wherein the method further comprises removing the steerable medical device from the jugular vein and sealing the opening.

14. A method for renal nerve modulation, the method comprising:
providing a steerable ablation guidewire having one or more ablation members coupled thereto;
advancing the steerable ablation guidewire through a renal vein to a position adjacent to a kidney;
advancing the steerable ablation guidewire through a vessel wall of the renal vein and into body tissue positioned adjacent to a renal artery;
steering the steerable ablation guidewire around at least a portion of the renal artery; and
ablating renal nerves disposed adjacent to the renal artery with the one or more ablation members.

15. The method of claim 14, wherein advancing the steerable ablation guidewire through a renal vein to a position adjacent to the kidney includes advancing the steerable ablation guidewire through a jugular vein.

16. The method of claim 14, wherein advancing the steerable ablation guidewire through a renal vein to a position adjacent to the kidney includes advancing the steerable ablation guidewire through the vena cava.

17. The method of claim 14, wherein advancing the steerable ablation guidewire through a renal vein to a position adjacent to the kidney includes forming an opening in a jugular vein, and wherein the method further comprises removing the steerable ablation guidewire from the jugular vein and sealing the opening.

18. A method for renal nerve modulation, the method comprising:
providing a steerable guidewire;
advancing the steerable guidewire through a renal vein to a position adjacent to a kidney;
advancing the steerable guidewire through a vessel wall of the renal vein and into body tissue positioned adjacent to a renal artery;
steering the steerable guidewire around at least a portion of the renal artery;
advancing an ablation catheter over the steerable guidewire and around at least the portion of the renal artery, wherein the ablation catheter has one or more ablation members coupled thereto; and
ablating renal nerves disposed adjacent to the renal artery with the one or more ablation members.

19. The method of claim 18, wherein advancing the steerable guidewire through a renal vein to a position adjacent to the kidney includes advancing the steerable guidewire through a jugular vein, the vena cava, or both.

20. The method of claim 18, wherein advancing the steerable guidewire through a renal vein to a position adjacent to the kidney includes forming an opening in a jugular vein, and wherein the method further comprises removing the steerable guidewire from the jugular vein and sealing the opening.
